(19) United States
(12) Patent Application Publication
(10) Pub. No.: US 2024/0325089 A1
(43) Pub. Date: Oct. 3, 2024

(54) SYSTEMS AND METHODS FOR NAVIGATION AND IDENTIFICATION DURING ENDOSCOPIC SURGERY

(71) Applicant: Vanderbilt University, Nashville, TN (US)

(72) Inventors: Nicholas L. Kavoussi, Nashville, TN (US); Ipek Oguz, Nashville, TN (US); Zachary Stoebner, Nashville, TN (US); Ayberk Acar, Nashville, TN (US); Jie Ying Wu, Nashville, TN (US); Daiwei Lu, Nashville, TN (US)

(73) Assignee: Vanderbilt University, Nashville, TN (US)

(21) Appl. No.: 18/622,134

(22) Filed: Mar. 29, 2024

Related U.S. Application Data
(60) Provisional application No. 63/455,626, filed on Mar. 30, 2023.

(51) Int. Cl. A61B 34/20; G06T 7/11; G06T 19/20

(52) U.S. Cl. CPC A61B 34/20; G06T 7/11; G06T 19/20; A61B 2034/2059; G06T 2207/10081; G06T 2207/30084

(57) ABSTRACT

Systems and methods for navigation and identification for endoscopic kidney surgery may include generating a map of an internal space of a patient's collecting system, including segmentation preoperative CT scans, using localization and three-dimensional reconstruction techniques on endoscopic video to create a point cloud, and registering the point cloud to the segmented CT scans. The systems and methods may include tracking a tip of the endoscope during the endoscopic kidney surgery using localization and three-dimensional reconstruction techniques. The systems and methods may include identifying and tracking kidney stones during the endoscopic kidney surgery using computational models.

### CROSS-REFERENCES TO RELATED APPLICATIONS

This application is a non-provisional of and claims priority to U.S. Provisional Application No. 63/455,626, filed Mar. 30, 2023, entitled SYSTEMS AND METHODS FOR NAVIGATION AND IDENTIFICATION DURING ENDOSCOPIC SURGERY, all of which is hereby incorporated by reference in its entirety.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### STATEMENT REGARDING FEDERALLY SPONSORED RESEARCH OR DEVELOPMENT

Not Applicable.

### BACKGROUND

The present disclosure generally relates to robotic surgery, and more particularly to systems and methods for navigation and identification during endoscopic kidney surgery.

Each year, over 100,000 patients in the United States undergo an endoscopic surgery for kidney stone disease. Additionally, approximately 15,000 patients in the United States are diagnosed with upper tract urothelial carcinoma (UTUC), which is a cancer that arises in the kidney. A surgeon usually performs an endoscopic surgery through the patient's ureter (ureteroscopy) or percutaneously through the flank into the patent's kidney (percutaneous nephrolithotomy) with a flexible endoscope in order to treat the kidney stones or UTUC. During surgery, video from the endoscope is displayed on a screen, enabling the surgeon to maneuver the endoscope and visualize the patient's renal collecting system. Patients' renal collecting systems are complex, having several branched renal calyces that come together to funnel urine into the renal pelvis and ureter. Kidney stones can exist in any part of the renal collecting system, and UTUC tumors can also be located in various locations of the collecting system. Once kidney stones are located in a patient's collecting system, they are treated with a combination of laser fragmentation or extraction. Similarly, once a UTUC tumor is located, it is treated using instruments at the end of the endoscope.

Unfortunately, approximately 25% of patients that undergo kidney stone surgery have a repeat surgery for residual stone fragments, which can lead to obstruction, pain, kidney injury, and urinary tract infections. Also, during endoscopic surgery to treat UTUC, if the surgeon misses a UTUC tumor and it is left untreated, the tumor can worsen and even develop into a metastatic disease. Several challenges lead to incomplete stone or tumor treatment, which can cause the need for repeated surgery.

First, successful surgery usually involves the surgeon having to visualize the entire renal collecting system and locate all kidney stones or tumors during treatment. It is difficult to for a surgeon to navigate the patient's three-dimensional anatomy using only preoperative, two-dimensional axial computerized tomography (CT) images as his or her guide. Second, several types of objects frequently obscure the already limited field of view during endoscopic surgery. Such objects can include blood, bubbles, kidney stone debris, blood clots, and other debris. Such limited view makes kidney stones and tumors difficult to identify and prevents navigation through the patient's anatomy. Third, during the surgery, kidney stones can fragment and disperse throughout the patient's anatomy, further complicating intraoperative tracking. These inherent limitations of endoscopic treatment prevent many surgeons from achieving a complete stone-free and tumor-free status during the first endoscopic kidney surgery.

What is needed, then, are systems and methods for navigation and identification during endoscopic kidney surgery.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

The present disclosure provides for a method of navigating an anatomical region. The method may include generating a map of an internal space of an anatomical region. Generating the map of the internal space of the anatomical region may include segmenting the anatomical region from a preoperative CT scan, generating a three-dimensional reconstruction of the anatomical region from video received from an endoscope, and registering the three-dimensional reconstruction to the CT scan to generate a three-dimensional map of the anatomical region.

In some embodiments, the method further includes tracking a tip of an endoscope within the anatomical region. Tracking the tip of the endoscope within the anatomical region may include initializing a location and orientation of the tip of the endoscope on the three-dimensional map of the anatomical region, receiving endoscopic video from the endoscope, and updating the location and orientation of the tip of the endoscope based on applying one or more localization and three-dimensional reconstruction techniques to the received endoscopic video.

In some embodiments, the method further includes identifying and tracking an anatomical feature of an anatomical region. Identifying and tracking the anatomical feature of the anatomical region may include generating training, validation, and testing datasets, wherein the training, validation, and testing datasets each include a plurality of images, where one or more images of the plurality of images are annotated with a location of the anatomical feature of the anatomical region in the image. Such identifying an tracking may further include training a computational model, which may include steps such as training the computational model on the training dataset using forward propagation and loss computation to adjust one or more parameters of the computational model, validating the computational model on the validation dataset, receiving a frame of a video feed from an endoscope, and inputting the frame into the computational model. Further, such identifying and tracking may include generating, via the computational model, an output, where the output includes data indicating one or more locations of the frame containing the anatomical feature, and adjusting a visual display that includes the frame with an overlay based on the data indicating one or more locations of the frame containing the anatomical feature.

In particular, the present disclosure may provide for a navigational system for use during endoscopic kidney surgery with the potential to improve stone-free and tumor-free rates and prevent recurrent stone surgeries, stone complications, or worsening tumors. The navigational system makes stone and tumor localization and tracking within a patient's renal collecting system easier and more accurate for the operator. Such navigation systems include at least three aspects: (1) generating a three-dimensional map of the internal space of a patient's renal anatomy; (2) real-time tracking of the tip of an endoscope in the generated map during endoscopic kidney surgery; and (3) identifying and tracking kidney stones (and other features) during endoscopic stone surgery.

Such systems and methods for use during endoscopic kidney surgery may include generating a three-dimensional, navigational map of a patient's collecting system anatomy. The systems and methods may segment preoperative computerized tomography (CT) images obtained during clinical care to create a three-dimensional navigational map for use during endoscopic surgery. The systems and methods may integrate one or more kidney locations by registering the endoscopic surgical video to one or more segmented CT images. The systems and methods may display the navigational mapping of the renal collecting system and one or more kidney stones or tumors in real-time on the visual display. The three-dimensional navigational map may be displayed with respect to the current endoscope position. The three-dimensional navigational map may display a global position of the endoscope, the kidney stones, the tumors, or other objects, with respect to the renal collecting system.

Such systems and methods for use during endoscopic kidney surgery may include real-time tracking of the tip of an endoscope in the three-dimensional navigational map during endoscopic kidney surgery. A system may use one or more localization and three-dimensional reconstruction techniques based on one or more images received from the endoscope tip's camera to adjust a visual display to update the location and orientation of the tip on the three-dimensional navigational map.

Such systems and methods for use during endoscopic kidney surgery may include automatic, real-time segmentation and tracking of kidney stones during endoscopic stone surgery. This aspect may include creating a real-time video overlay over a video feed from an endoscope that may outline kidney stones on the visual display. This may include training computer vision models using data from endoscopic surgical videos. This overlay software may integrate with current endoscopic surgery displays in the operating room.

Thus, the present disclosure describes a useable, functional, high-fidelity, and validated navigational system for procedures such as endoscopic kidney surgery. Such a system may include a software integration to current endoscopic surgical cameras. Thus, one or more existing endoscopic surgical systems may immediately benefit from the present disclosure. In this way, the systems and methods of the present disclosure may facilitate improved stone-free and tumor-free surgery rates and mitigate repeat interventions or complications, benefiting patients, surgeons, and society.

Numerous other objects, advantages and features of the present disclosure will be readily apparent to those of skill in the art upon a review of the following drawings and description of various embodiments.

### BRIEF DESCRIPTION OF THE DRAWINGS

FIG. 1 is a schematic block diagram illustrating one embodiment of a system for navigation and identification during endoscopic kidney surgery.

FIG. 2 is a flowchart diagram illustrating one embodiment of a method for navigation during endoscopic kidney surgery.

FIG. 3A is a flowchart diagram illustrating one embodiment of a process for generating a three-dimensional structure as part of the method of FIG. 2.

FIG. 3B is a flowchart diagram illustrating one embodiment of a process for generating a three-dimensional reconstruction as part of the method of FIG. 2.

FIG. 3C is a perspective view illustrating one embodiment of a three-dimensional map generated by the method of FIG. 2.

FIG. 4 is a front view illustrating one embodiment of a user interface for a system for navigation and identification during endoscopic kidney surgery.

FIG. 5 is a flowchart diagram illustrating one embodiment of a method for identifying a kidney stone during endoscopic kidney stone surgery.

FIG. 6A is a front view illustrating one embodiment of an image used in training data in a system for identifying kidney stones during endoscopic kidney surgery.

FIG. 6B is a front view illustrating one embodiment of an annotated image used in training data in a system for identifying kidney stones during endoscopic kidney surgery.

FIG. 7A is a front view illustrating one embodiment of an input image used by a computational model to generate a prediction in a system for identifying kidney stones during endoscopic kidney surgery.

FIG. 7B is a front view illustrating one embodiment of a visual representation of a prediction generated by a computational model in a system for identifying kidney stones during endoscopic kidney surgery.

FIG. 7C is a front view illustrating one embodiment of a user interface displaying an overlay to a video feed of an endoscope in a system for identifying kidney stones during endoscopic kidney surgery.

FIG. 8A is a front view illustrating one embodiment of an input image used by a computational model to generate a prediction in a system for identifying kidney stones during endoscopic kidney surgery.

FIG. 8B is a front view illustrating one embodiment of a visual representation of a prediction generated by a computational model in a system for identifying kidney stones during endoscopic kidney surgery.

FIG. 8C is a front view illustrating one embodiment of a user interface displaying an overlay to a video feed of an endoscope in a system for identifying kidney stones during endoscopic kidney surgery.

FIG. 9A is a front view illustrating one embodiment of an input image used by a computational model to generate a prediction in a system for identifying UTUC tumors during endoscopic kidney surgery.

FIG. 9B is a front view illustrating one embodiment of a visual representation

of a prediction generated by a computational model in a system for identifying UTUC tumors during endoscopic kidney surgery.

FIG. 9C is a front view illustrating one embodiment of a user interface displaying an overlay to a video feed of an endoscope in a system for identifying UTUC tumors during endoscopic kidney surgery.

### DETAILED DESCRIPTION

While the making and using of various embodiments of the present disclosure are discussed in detail below, it should be appreciated that the present disclosure provides many applicable inventive concepts that are embodied in a wide variety of specific contexts. The specific embodiments discussed herein are merely illustrative of specific ways to make and use the disclosure and do not delimit the scope of the disclosure. Those of ordinary skill in the art will recognize numerous equivalents to the specific apparatus and methods described herein. Such equivalents are considered to be within the scope of this disclosure and are covered by the claims.

### Terminology

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

Reference throughout this specification to “one embodiment,” “an embodiment,” “another embodiment,” or similar language means that a particular feature, structure, or characteristic described in connection with the embodiment is included in at least one embodiment. Thus, appearances of the phrases “in one embodiment,” “in an embodiment,” “in some embodiments,” and similar language throughout this specification may, but do not necessarily, all refer to the same embodiment, but mean “one or more but not necessarily all embodiments” unless expressly specified otherwise.

The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. As used herein, the term “a,” “an,” or “the” means “one or more” unless otherwise specified. The term “or” means “and/or” unless otherwise specified.

Multiple elements of the same or a similar type may be referred to as “Elements 102(1)-(n)” where n may include a number. Referring to one of the elements as “Element 102” refers to any single element of the Elements 102(1)-(n). Additionally, referring to different elements “First Elements 102(1)-(n)” and “Second Elements 104(1)-(n)” does not necessarily mean that there must be the same number of First Elements as Second Elements and is equivalent to “First Elements 102(1)-(n)” and “Second Elements (1)-(m)” where m is a number that may be the same or may be a different number than n.

As used herein, the term “computing device” may include a desktop computer, a laptop computer, a tablet computer, a mobile device such as a mobile phone or a smart phone, a smartwatch, a gaming console, an application server, a database server, or some other type of computing device. A computing device may include a physical computing device or may include a virtual machine (VM) executing on another computing device. A computing device may include a cloud computing system, a distributed computing system, or another type of multi-device system.

As used herein, the term “data network” may include a local area network (LAN), wide area network (WAN), the Internet, or some other network. A data network may include one or more routers, switches, repeaters, hubs, cables, or other data communication components. A data network may include a wired connection or a wireless connection.

As used herein, the term “computing platform” or “platform” may include a computing environment where a portion of software can execute. A computing platform may include hardware on which the software may execute. The computing platform may include an operating system. The computing platform may include one or more software applications, scripts, functions, or other software. The computing platform may include one or more application programming interfaces (APIs) by which different portions of the software of the platform may communicate with each other or invoke functions. The computing platform may include one or more APIs by which it may communicate with external software applications or by which external software applications may interact with the platform. The computing platform may include a software framework. The computing platform may include one or more VMs. The software platform may include one or more data storages. The software platform may include a client application that executes on an external computing device and that interacts with the platform in a client-server architecture.

As used herein, the term “data storage” may include a tangible device that retains and stores data. Such device may include an electronic storage device, a magnetic storage device, an optical storage device, an electromagnetic storage device, a semiconductor storage device, or any suitable combination of the foregoing. A non-exhaustive list of more specific examples of the devices may include a portable computer diskette, a random access memory (RAM), a read-only memory (ROM), an erasable programmable read-only memory (EPROM or flash memory), a static random access memory (SRAM), a hard disk drive (HDD), a solid state drive, a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. “Data storage,” in some embodiments, may include a data structure that stores data, and the data structure may be stored on a tangible data storage. Such data storage may include a file system, a database, cloud storage, a data warehouse, a data lake, or other data structures configured to store data.

As used herein, the terms “determine” or “determining” may include a variety of actions. For example, “determining” may include calculating, computing, processing, deriving, looking up (e.g., looking up in a table, a database or another data structure), ascertaining, or other actions. Also, “determining” may include receiving (e.g., receiving information or data), accessing (e.g., accessing data in a memory, data storage, distributed ledger, or over a network), or other actions. Also, “determining” may include resolving, selecting, choosing, establishing, or other similar actions.

As used herein, the terms “provide” or “providing” may include a variety of actions. For example, “providing” may include generating data, storing data in a location for later retrieval, transmitting data directly to a recipient, transmitting or storing a reference to data, or other actions. “Providing” may also include encoding, decoding, encrypting, decrypting, validating, verifying, or other actions.

As used herein, the term “access,” “accessing”, and other similar terms may include a variety of actions. For example, accessing data may include obtaining the data, examining the data, or retrieving the data. Providing access or providing data access may include providing confidentiality, integrity, or availability regarding the data.

As used herein, the term “message” may include one or more formats for communicating (e.g., transmitting or receiving) information or data. A message may include a machine-readable collection of information such as an Extensible Markup Language (XML) document, fixed-field message, comma-separated message, or another format. A message may, in some implementations, include a signal utilized to transmit one or more representations of information or data.

As used herein, the term “user interface” (also referred to as an interactive user interface, a graphical user interface or a UI), may refer to a computer-provided interface including data fields or other controls for receiving input signals or providing electronic information or for providing information to a user in response to received input signals. A user interface may be implemented, in whole or in part, using technologies such as hyper-text mark-up language (HTML), a programming language, web services, or rich site summary (RSS). In some implementations, a user interface may be included in a stand-alone client software application configured to communicate in accordance with one or more of the aspects described.

As used herein, the term “modify” or “modifying” may include several actions. For example, modifying data may include adding additional data or changing the already-existing data. As used herein, the term “obtain” or “obtaining” may also include several types of action. For example, obtaining data may include receiving data, generating data, designating data as a logical object, or other actions.

As used herein, the term “data object” may include a logical container for data. A data object may include an instance of an object in a software application implemented with an object-oriented programming language. A data object may include data formatted in an electronic data interchange (EDI) format, such as an extensible Markup Language (XML) object, a JavaScript Object Notation (JSON) object, or some other EDI-formatted object. A data object may include one or more functions that may manipulate the data of the data object. For example, a data object may include the functions or methods of an object in a software application implemented with an object-oriented programming language.

### Overview

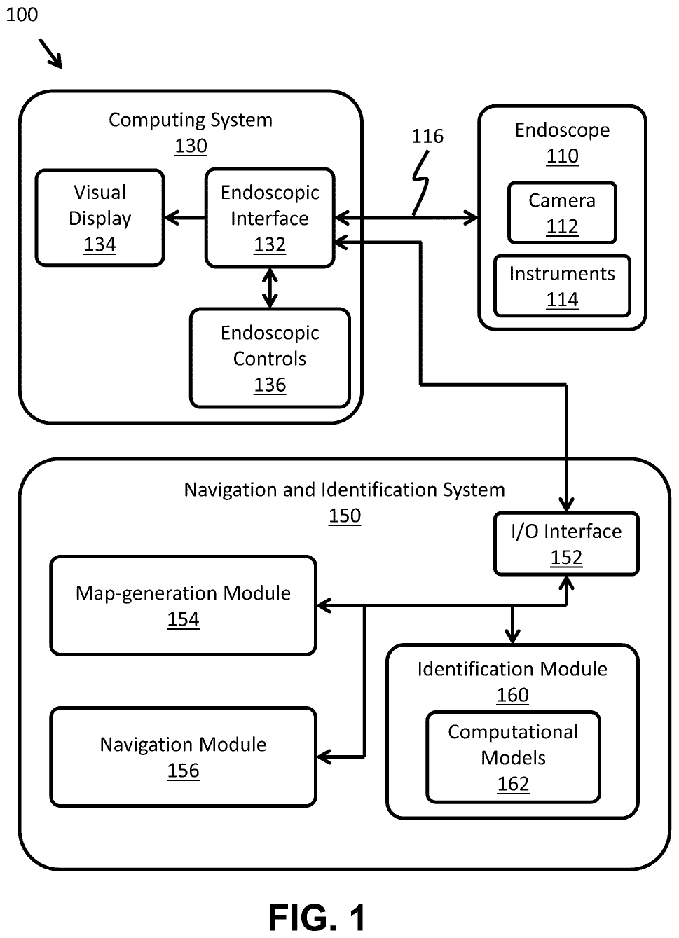

FIG. 1 depicts one embodiment of a system 100 for navigating an anatomical region and identifying one or more features within the anatomical region. For instance, the system 100 may be configured for navigating an anatomical region and/or identifying one or more features within the anatomical region. In some embodiments, the anatomical region is a collecting system of a kidney, and the feature of the anatomical region is a kidney phenomenon, which may be a kidney stone, a kidney stone fragment, a tumor, and so on.

The system 100 may include an endoscope 110. The endoscope 110 may include an endoscope used in performing a healthcare procedure, such as kidney surgery. The endoscope 110 may include a camera 112 disposed on the tip of the endoscope 110. The endoscope 110 may include one or more instruments 114 (e.g., an illumination source, a manipulator, a laser) disposed on or near the tip of the endoscope 110. As suggested above, the system 100 may be configured to perform a method of navigating an anatomical region and/or identifying one or more features within the anatomical region. As discussed in greater detail below, the method may include: (1) generating a map of an internal space of an anatomical region (e.g., a patient's collecting system), (2) tracking a tip of the endoscope 110 within the anatomical region, and/or (3) identifying and tracking a feature of the anatomical region (e.g., a kidney phenomenon).

In one embodiment, the system 100 may include a computing system 130 (e.g., a surgical computing system). The computing system 130 may include a computing device used in performance of a healthcare procedure, such as an endoscopic kidney stone surgery. The computing system 130 may control the endoscope 110 and may display a video feed from the endoscope 110. The endoscope 110 may connect to the computing system 130 via a tube 116, which may include one or more channels for wires, the instruments 114, or other endoscopic hardware. The computing system 130 may include an endoscopic interface 132, which may include software or hardware that may interface between the endoscope 110 and other components of the computing system 130. The computing system 130 may include a visual display 134 (e.g., a computer monitor). The computing system 130 may include endoscopic controls 136, which may include hardware or software that a user can manipulate to control the movement or instruments 114 of the endoscope 110 (e.g., a keyboard, mouse, joystick).

The system 100 may include a navigation and identification system 150. The navigation and identification system 150 may include a computing device. The navigation and identification system 150 may be in data communication with the computing system 130. The navigation and identification system 150 may include an input/output (I/O) interface 152. The I/O interface may include hardware or software that may interface between the navigation and identification system 150 and the endoscope 110 or the computing system 130. The navigation and identification system 150 may include a map-generation module 154. The map-generation module may generate a map (e.g., a three-dimensional map) of an anatomical region (such as a patient's collecting system of a kidney) based on data received from a video feed of the endoscope 110, computed tomography (CT) scans, or other data. The navigation and identification system 150 may include a navigation module 156. The navigation module may track the location of the tip of the endoscope 110 within the generated three-dimensional map of the patient's collecting system anatomy based on movement in a video feed of the endoscope 110 or based on other data. The navigation and identification system 150 may include an identification module 160. The identification module 160 may identify features of an anatomical region such as kidney stones, kidney stone fragments, or other anatomical phenomena from the video feed of the endoscope 110. The identification module 160 may include one or more computational models 162 (sometimes referred to herein simply as “models”). A computational model 162 may include a machine learning, artificial intelligence (AI), or statistical model, or some other type of model that may use the endoscope 110 video feed as input in order to perform the identification.

Further details of one or more embodiments of the system 100 are now discussed. In some embodiments, the endoscope 110 may include an endoscope used in performing endoscopic kidney surgery or some other type of surgery. In various other embodiments, the endoscope 110 includes an endoscope configured for other applicable healthcare procedures. The endoscope 110 may include a camera 112 disposed on the tip of the endoscope 110. The camera 112 may stream or record video during the procedure. The endoscope 110 may include one or more instruments 114. An instrument 114 may include an illumination source such as a light. An instrument 114 may include a manipulator used to clasp an object. An instrument 114 may include forceps, a cutter, burs, a brush, a tube set, a tissue stapler, a ligation device, a suturing system, or some other instrument. An instrument 114 may include a laser. A laser may include a laser configured to break up kidney stones, configured to ablate a tumor or surrounding tissue, or some other type of laser. One or more of the instruments 114 may be disposed on or near the tip of the endoscope 110. The tube 116 may include one or more channels. A channel may contain a portion of an instrument 114, a wire (e.g., from the camera 112 to the endoscopic interface 132), or other endoscope 110 hardware. The tube 116 may be various lengths or widths or may be flexible.

In one embodiment, the computing system 130 may include an endoscopic interface 132. The endoscopic interface 132 may include software or hardware that may interface between the endoscope 110 and other components of the computing system 130. The endoscopic interface 132 may include a digital visual interface (DVI) connection disposed between the endoscope's 110 camera 112 and a video capture card (which may also form part of the endoscopic interface 132). The endoscopic interface 132 may be in data communication with other portions of the computing system 130, such as the visual display 134 or the endoscopic controls 136. The endoscopic interface 132 may be in data communication with other computing devices, such as the navigation and identification system 150.

In one embodiment, the visual display 134 of the computing system 130 may include a computer monitor, a touch screen, a tablet, or some other device capable of providing a visual output. The visual display 134 may be disposed in a location where an operator (e.g., a surgeon) performing the procedure using the endoscope 110 may view the visual display 134. The endoscopic controls 136 may include one or more devices used to control the movement of the endoscope 110 or the instruments 114 of the endoscope 110. The endoscopic controls 136 may include a keyboard, a mouse, a joystick, a knob, a turn-wheel, a button, or other input controls. The endoscopic controls 136 may be disposed in a location of the operator performing the procedure.

In one embodiment, the navigation and identification system 150 may include a computing device separate from the computing device of the computing system 130. In some embodiments, the navigation and identification system 150 may include software included on the computing device of the computing system 130. The I/O interface 152 may include a DVI connection disposed between the camera 112 and other portions of the navigation and identification system 150. The I/O interface 152 may include other hardware or software that allows the computing system 130 and one or more components of the navigation and identification system 150 to be in data communication. Further details regarding the map-generation module 154, the navigation module 156, and the identification module 160 are given below.

### Generating a Three-Dimensional Map of the Internal Space of an Anatomical Region

As an overview, the navigation and identification system 150 may include a map-generation module 154 that may include a three-dimensional navigational system for procedures (such as endoscopic kidney surgery) that may integrate a three-dimensional segmentation of a patient's collecting system from preoperative imaging and register it to the endoscopic video via one or more localization and three-dimensional reconstruction techniques.

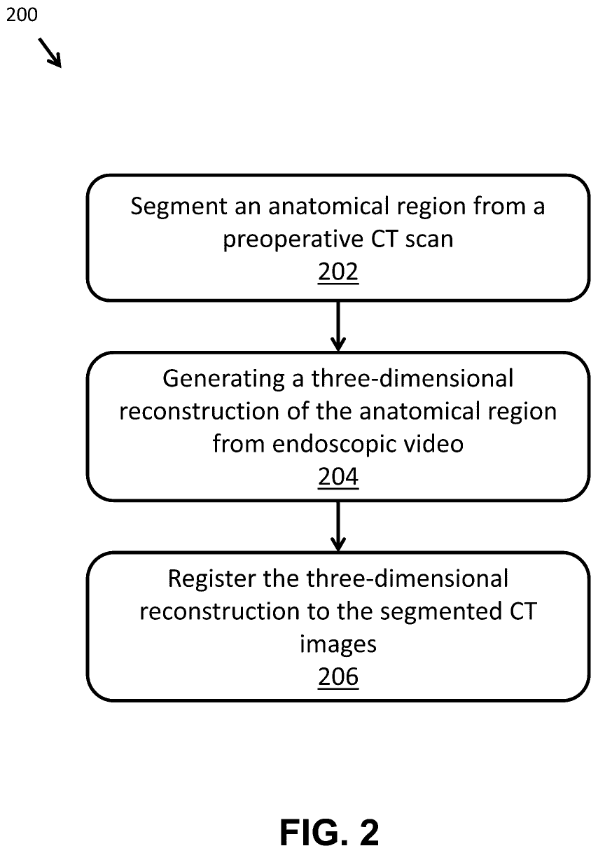

FIG. 2 depicts one embodiment of a method 200. The method 200 may include a method of generating a map of an internal space of an anatomical region (such as a patient's collecting system). The method 200 may include segmenting the anatomical region from one or more preoperative CT scans (step 202). The method 200 may include generating a three-dimensional reconstruction of the anatomical region from video received from the endoscope 110 (e.g., endoscopic video of the patient's collecting system) (step 204). The method 200 may include registering the three-dimensional reconstruction to the segmented CT scan to generate a three-dimensional map of the anatomical region (step 206). For instance, step 206 may include registering the three-dimensional reconstruction generated from step 204 to the three-dimensional structure generated from step 202. In some embodiments, the map-generating module 154 may perform one or more of the steps 202-206 of the method 200.

In one embodiment, segmenting the anatomical region from one or more preoperative CT scans (step 202) may include the map-generating module 154 receiving the data of the one or more CT scans. The data may include image files or other data. The data may be received via the I/O interface 152 and stored in a data storage on the navigation and identification system 150 or in a data storage that is in data communication with the navigation and identification system 150. Segmenting the one or more CT scans may include the map-generating module 154 inputting the data into image segmentation software such as 3D Slicer provided by The Brigham and Women's Hospital, Inc. In some embodiments, the image-segmenting functionality may be incorporated into the map-generating module 154. Step 202 may include the map-generation module 154 performing one or more three-dimensional modeling operations on the segmented CT scan to generate data representing a three-dimensional structure of the segmented CT scan.

In some embodiments, generating a three-dimensional reconstruction of the anatomical region (step 204) may include the map-generation module 154 accepting the sequence of endoscopic video frames as input and generating a three-dimensional point cloud. The map-generation module 154 may generate the point cloud using one or more localization and three-dimensional reconstruction techniques. A localization and three-dimensional reconstruction technique may include an algorithm, method, procedure, function, or other technique for estimating a three-dimensional structure based on image data. A localization and three-dimensional reconstruction technique may include a localization technique or a three-dimensional reconstruction technique. A localization and three-dimensional reconstruction technique may include structure from motion, simultaneous localization and mapping (SLAM), or some other type of localization and three-dimensional reconstruction technique. A localization and three-dimensional reconstruction technique may include algorithms, libraries, software, or machine learning models, such as a COLMAP (with scale-invariant feature transform) pipeline provided by ETH Zurich and UNC Chapel Hill, SuperPoint and SuperGlue (i.e., Pixel-Perfect without refinement, a neural network-based method) deep-learning models, or a Pixel-Perfect (with refinement) deep-learning model. The map-generation module 154 may use feature extraction and matching from the video feed to establish correspondences between points on the image plan and the world coordinate system.

In one embodiment, the machine learning models of the map-generation module 154 used to generate the point cloud may include machine learning models that have been trained on annotated CT scans. The CT scans may have been annotated with one or more anatomical features such as renal collecting system anatomy, UTUC tumors, or other phenomena encountered in the anatomical region.

In some embodiments, the point cloud may then be registered to a three-dimensional segmentation of the anatomical region derived from CT data (step 206) to create a three-dimensional map of the anatomical region. The registration may include using iterative closest point registration. Registration of the point cloud may include using a three-dimensional analysis software library (such as Open3D provided by Qian-Yi Zhou and Jaesik Park and Vladlen Koltun). Registration may include using a three-dimensional iterative closest point (ICP) registration to align the point cloud to the three-dimensional structure from step 202. This may allow for the acquisition of three-dimensional coordinates of the endoscope 110 at each position.

In some embodiments, the I/O interface 152 or the map-generation module 154 may provide the video feed to the identification module 160 during the registration process, and the identification module 160 may identify one or more anatomical features (e.g., kidney stones, kidney stone fragments, clots, tumors, or other anatomical phenomena as explained further below). The map-generation module 154 may mark or annotate the three-dimensional map with the locations of the identified anatomical features. The map-generation module 154 may display one or more navigational directions to a location on the map. The navigational directions may include one or more arrows pointing to the location, either on the map or on the video feed.

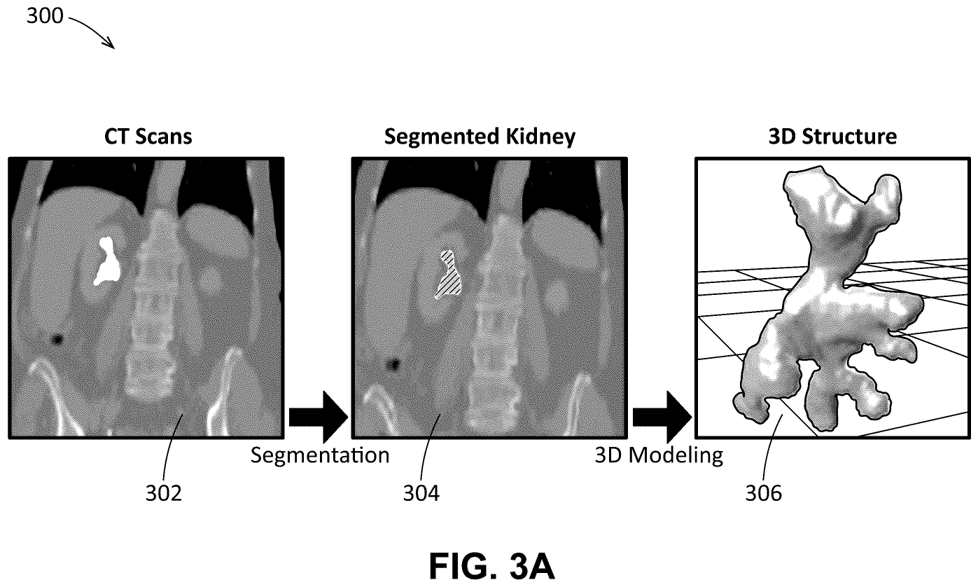

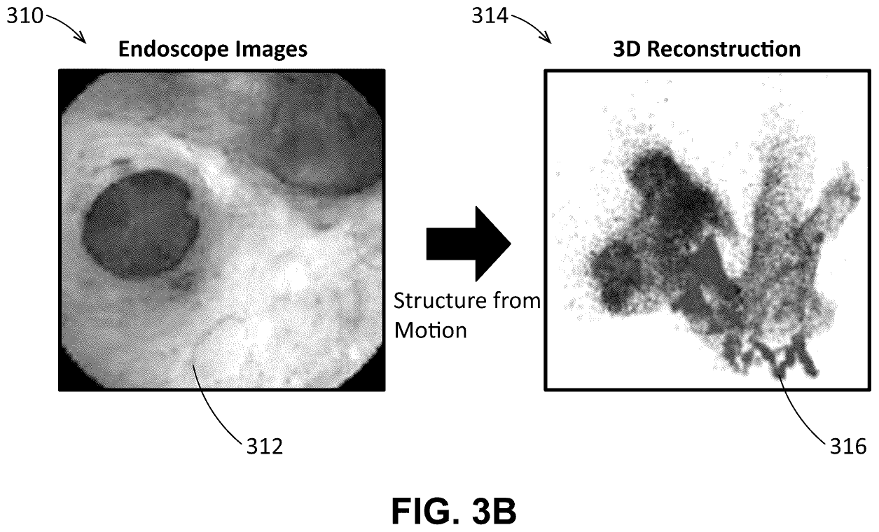

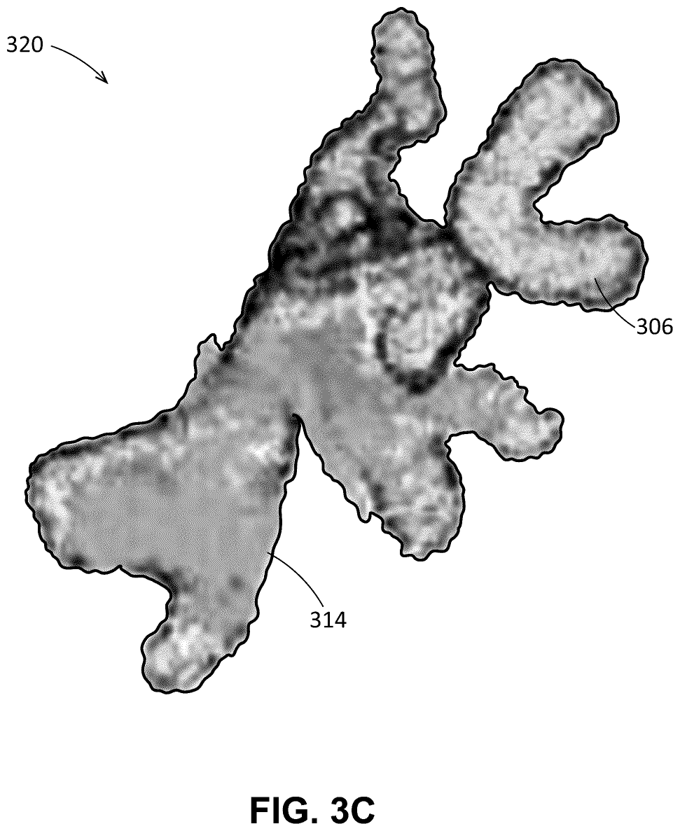

FIG. 3A depicts one embodiment of the process 300 of segmenting the anatomical region from one or more preoperative CT scan (step 202). For instance, FIG. 3A depicts an embodiment of the process 300 where such anatomical region is a patient's collecting system, as applicable for endoscopic kidney surgery. The map-generation module 154 may receive one or more CT scans 302. The map-generation module 154 may perform image segmentation on the one or more CT scans 302 to generate the segmented kidney data 304. The map-generation module 154 may perform one or more three-dimensional modeling operations to generate data representing a three-dimensional structure 306. While this three-dimensional structure 306 generated from the one or more CT scans may be a good starting point, it may be limited due to the limited amount of information contained within the one or more CT scans.

FIG. 3B depicts one example embodiment of the process 310 of generating a three-dimensional reconstruction of the anatomical region from endoscopic video of the anatomical region (step 204). For instance, FIG. 3B depicts an embodiment of the process 310 where such anatomical region is a patient's collecting system, as applicable for endoscopic kidney surgery. The map-generation module 154 may receive one or more frames of a video feed of the endoscope 110. These one or more frames may be sequential. The one or more frames may form part of one or more endoscopic images 312. The map-generation module 154 may perform one or more localization and three-dimensional reconstruction techniques (such as structure from motion or SLAM operations) on the one or more endoscopic images 312 to generate data representing a three-dimensional reconstruction 314 of the patient's collecting system depicted in the endoscopic video. In the three-dimensional reconstruction 314, the one or more dots 316 may represent the pose of the tip of the endoscope 110 at some point during the streaming or recordation of the endoscopic video. The map-generation module 154 may be able to use the three-dimensional reconstruction 314 to provide more detail to the three-dimensional structure 306 generated from the one or more CT scans in step 202.

FIG. 3C depicts one embodiment of a three-dimensional map 320. The map-generation module 154 may generate the three-dimensional map 320 as part of the endoscopic video registration result of step 206. The three-dimensional map 320 may include the three-dimensional structure 306 generated from the CT scan segmentation of step 202 with the three-dimensional reconstruction 314 from the endoscopic video of step 204. Generating the three-dimensional map 320 may include registering the points of the three-dimensional reconstruction 314 to the three-dimensional structure 306.

In some embodiments, the method 200 may not include segmenting the anatomical region from one or more preoperative CT scan (step 202). Instead, the map-generation module 154 may only use the endoscopic images 312 to generate the three-dimensional reconstruction 314 (step 204), which then may be used to generate the three-dimensional map 320 (step 206). In some embodiments, step 202 may include using synthetic visualizations instead of preoperative CT scans, and step 206 may include matching frames from the endoscopic images 312 using localization and three-dimensional reconstruction techniques to generate the three-dimensional map 302.

Accordingly, the present disclosure provides for a method of navigating an anatomical region, which may include generating a map of an internal space of an anatomical region. As discussed above, generating the map of the internal space of the anatomical region may include segmenting the anatomical region from a preoperative CT scan, generating a three-dimensional reconstruction of the anatomical region from video received from an endoscope, and registering the three-dimensional reconstruction to the CT scan to generate a three-dimensional map of the anatomical region.

### REAL-TIME TRACKING OF ENDOSCOPE TIP DURING ENDOSCOPIC PROCEDURE

As an overview, the navigation and identification system 150 may include a navigation module 156. The navigation module 156 may track the tip of the endoscope 110 in real time during a procedure (such as endoscopic kidney surgery). The navigation module 156 may provide a visual output to a portion of the visual display 134 of the computing system 130. The visual output may include a three-dimensional map 320 showing an anatomical region (such as a patient's collecting system). The three-dimensional map 320 may include an icon indicating the endoscope's 110 tip's location and orientation. The three-dimensional map 320 may include icons indicating the location of anatomical features in the patient's collecting system (such as kidney stones or tumors). The visual output may include a picture-in-picture where the larger picture includes the video feed of the endoscope's 110 camera 112, and the smaller picture includes the three-dimensional map 320 with the icons. The visual output may include navigational directions, such as arrows pointing to one or more locations on the map or in the anatomical region. In one embodiment, the three-dimensional navigational map 320 may be displayed with respect to the current endoscope 110 tip position. In some embodiments, the three-dimensional navigational map 320 may display a global position of the endoscope tip 110, as well as anatomical features with respect to the anatomical region (such as kidney stones, tumors, or other objects, with respect to the renal collecting system).

In one embodiment, the navigation module 156 may determine the initial location and orientation of the endoscope's 110 tip within the three-dimensional map 320. In some embodiments, the navigation module 156 may estimate the initial position based on the assumption that the endoscope 110 usually enters through an anatomical pathway (such as the patient's ureter) and that there are a limited number of angles at which the endoscope's 110 tip could exit the pathway.

The navigation module 156 may determine the location and orientation of the endoscope 110 tip as it moves through the anatomical region and may update the tip's location and orientation on the three-dimensional map 320 in real time. The navigation module 156 may accept, as input, sequential frames of the endoscope's 110 camera's 112 video feed. The navigation module 156 may perform one or more localization and three-dimensional reconstruction techniques (e.g., structure from motion or SLAM) to determine an updated location or orientation of the endoscope's 110 tip. Such localization and three-dimensional reconstruction techniques may be similar to those described above in relation to step 204 of the method 200. In some embodiments, determining the location or orientation may include the navigation module 156 generalizing one or more SLAM models to a deformable scene from a template based on a segmented CT image. This may be converted into a triangle mesh using a marching cubes algorithm. The navigation module 156 may reconstruct the endoscopic procedural scene by matching key points in consecutive frames from the endoscopic video feed and estimate the deformation that would occur to match the reconstruction to the template. The navigation module 156 may match key points between frames and correct them using bundle adjustment to conform to the estimated deformation.

In one embodiment, to further reduce the computational burden of the navigation and identification system 150, the navigation module 156 may select a portion of the mesh to perform the template matching. The navigation module 156 may dynamically select the portion of the mesh to which the endoscope 110 frame is matched, based on the localization and the uncertainty. In some embodiments, domain-specific features may be important for accurate intracorporeal SLAM. In one or more embodiments, general-purpose features may result in the loss of endoscope 110 tracking in some frames, particularly during the traversal of some tubular sections of the renal collecting system. The navigation module 156 identifying anatomically-specific key points (via training of computational models on anatomically-specific training data, such as kidney-specific training data) may make the registration step of SLAM more robust. The navigation module 156 may incorporate identification of branching points in the anatomical region from the endoscope 110 video feed and match them to the three-dimensional map 320. In response to the navigation module 156 identifying a branch opening, the navigation module 156 may localize its algorithms, techniques, etc. to the current branch being traversed. This may provide the navigation module 156 global information about the location of the endoscope 110 within the three-dimensional map 320. The navigation module 156 may use this to ameliorate the error accumulation in iterative algorithms like SLAM.

In some embodiments, pre-processing the video feed from the endoscope 110 using filters designed for underwater use may improve the reconstruction by reducing the noise from fluid artifacts (e.g., bubbles). In one embodiment, the navigation module 156 may use a CycleGAN computational model (provided by Jun-Yan Zhu, Taesung Park, Phillip Isola, and Alexei A. Efros) or a Contrastive Unpaired Translation (CUT) model (provided by Taesung Park, Alexei A. Efros, Richard Zhang, Jun-Yan Zhu) to use a synthetic artifact synthesis model in reverse, allowing the navigation module 156 to remove fluid artifacts and debris to create “clean” images for registration purposes. The navigation module 156 may use anatomical features (such as tumors or other kidney phenomena) as landmarks to confirm that the correct branch was selected.

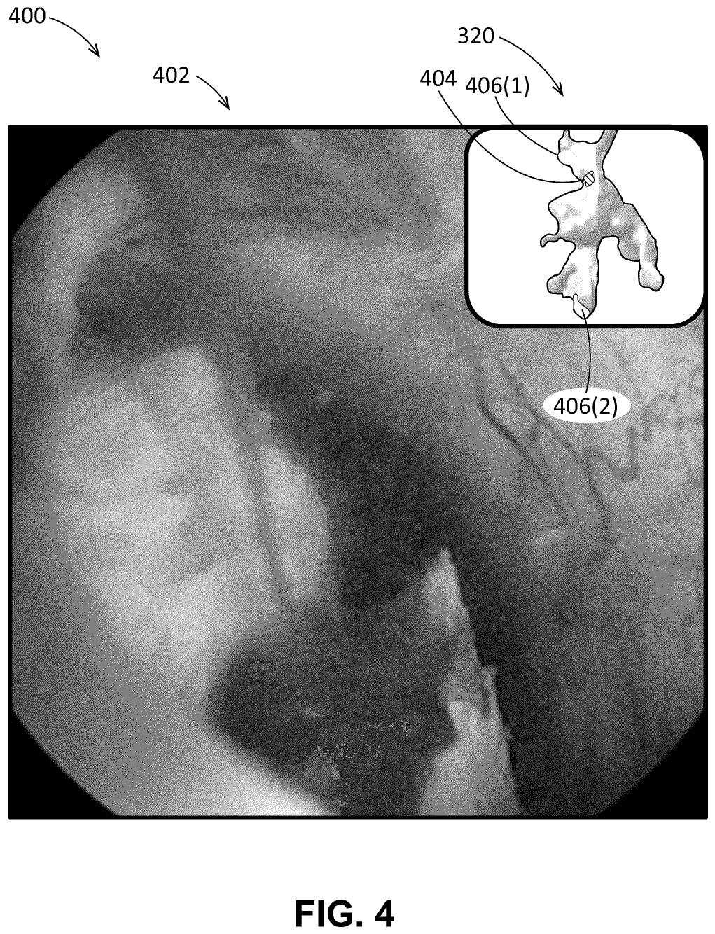

FIG. 4 depicts one embodiment of a user interface 400 displayable on the visual display 134. For instance, FIG. 4 depicts an embodiment of the user interface as applied to an endoscopic kidney surgery procedure. The user interface 400 may include a picture-in-picture layout showing the video feed from the endoscope 110 in the main picture 402 and the three-dimensional map 320 in the smaller picture. In some embodiments, the three-dimensional map 320 may include an icon 404 indicating the location or orientation of the endoscope 110 tip. The icon 404 may include an arrow that points in the direction the endoscope 110 tip is facing. The three-dimensional map 320 may include one or more icons 406 that indicate the location of some type of phenomena in the patient's collecting system. For example, as shown in FIG. 4, the three-dimensional map 320 may include two icons 406(1)-(2) that may indicate the locations of tumors in the patient's collecting system.

In one embodiment, a user of the computing system 130 may use the endoscopic controls 136 to move the endoscope 110 tip, and in response, the three-dimensional map 320 may update in real time to reflect the endoscope's 110 movement. In some embodiments, the three-dimensional map 320 may automatically rotate or reorient in response to the movement of the endoscope 110. In some embodiments, the user may rotate the map 320, zoom in or out, or perform other actions on the map using the endoscopic controls 136 or other controls of the computing system 130.

Accordingly, the present disclosure provides for a method of navigating an anatomical region, which may include tracking a tip of an endoscope within the anatomical region. As discussed above, tracking the tip of the endoscope within the anatomical region may include initializing a location and orientation of the tip of the endoscope on the three-dimensional map of the anatomical region, receiving endoscopic video from the endoscope, and updating the location and orientation of the tip of the endoscope based on applying one or more localization and three-dimensional reconstruction techniques to the received endoscopic video.

### Identifying and Tracking Anatomical Features Using Computational Models

As an overview, the navigation and identification system 150 may include a system for training a computer vision model for segmentation and tracking of anatomical features (such as kidney stones and stone fragments), using the trained model to identify such anatomical features, and integrating this information in the visual display during a procedure (such as endoscopic stone surgery). In one embodiment, such a system may include the identification module 160. As used herein, the term “tracking” may include temporal segmentation of an anatomical feature (and fragments thereof, such as when the anatomical feature is a kidney stone) throughout the video stream. Current endoscopic visibility during such procedures can be limited by blood and debris, which may interfere with a operator's ability to treat applicable anatomical features.

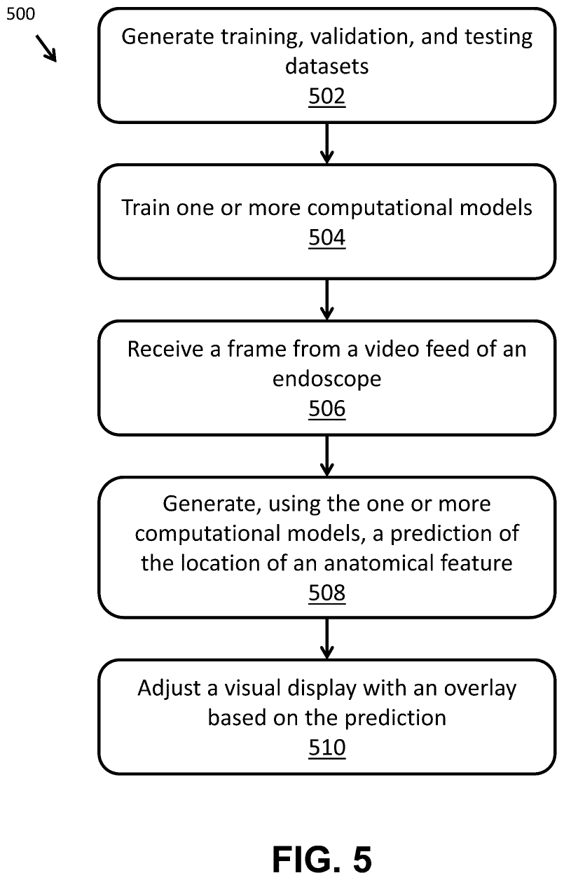

FIG. 5 depicts one embodiment of a method 500. The method 500 may include a method of identifying and tracking a feature of an anatomical region (such as kidney stones within a patient's collecting system) using computational models 162. The method 500 may include generating training, validation, and testing datasets (step 502). The method 500 may include training one or more computational models 162 using the training, validation, and testing datasets (step 504). The method 500 may include receiving a frame from a video feed of an endoscope 110 (step 506). The method 500 may include generating, using the one or more computational models 162, a prediction of the location of a kidney stone in the frame (step 508). The method 500 may include adjusting a visual display 134 with an overlay based on the prediction (step 510).

In one embodiment, the identification module 160 may be operable to generate a dataset for training, validation, and testing (step 502). Generating the dataset may include obtaining one or more images. The images may be provided from the I/O interface 152. For example, the I/O interface 152 may include a network interface, and the images may include image files received over a data network.

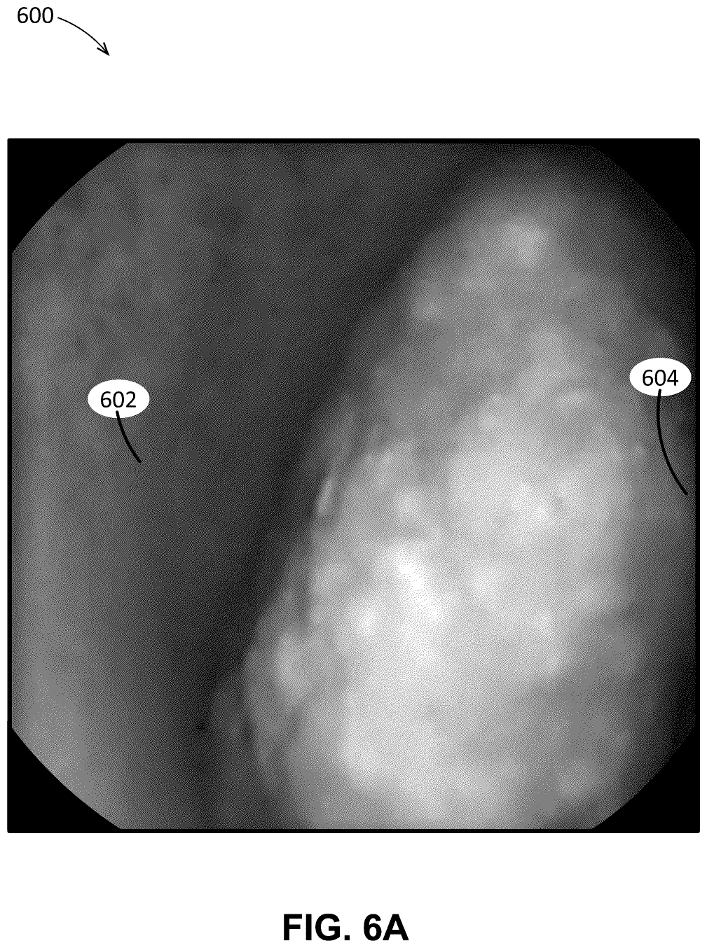

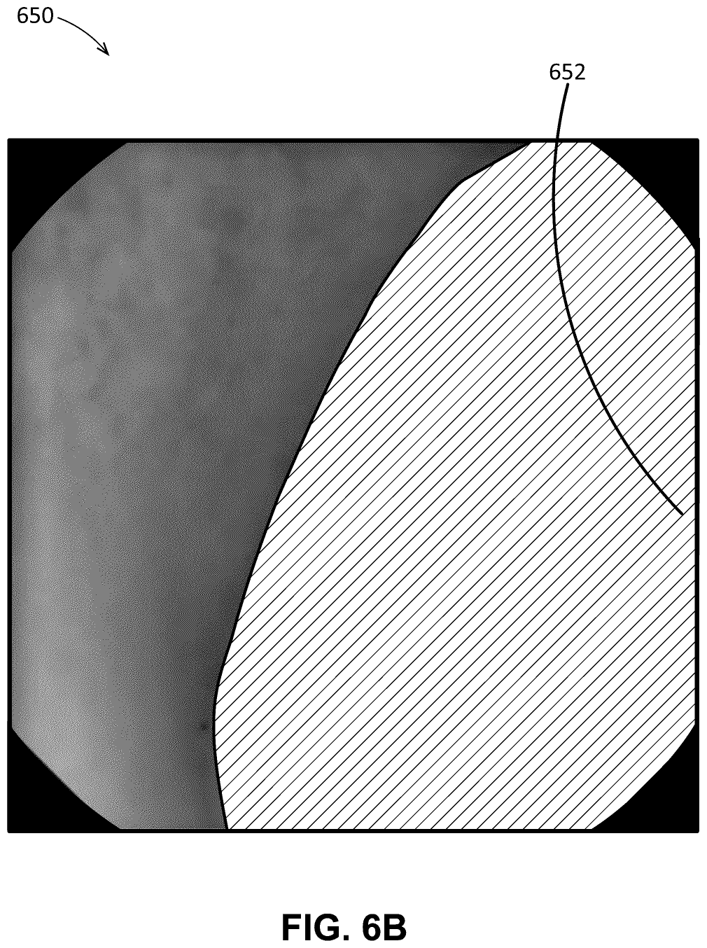

FIG. 6A depicts one embodiment of an image 600. An image 600 may include a frame from an endoscopic video, such as a ureteroscopy video. The image 600 may include a anatomical region 602, patient's collecting system anatomy. The image 600 may or may not include an anatomical feature 604 (such as a kidney stone or a kidney stone fragment) present in the image 600. Generating the dataset may include annotating an image 600 of the one or more images. Annotating an image 600 may include using a computer vision library to identify a contour in the image 600. The computer vision library may include OpenCV provided by the Open Source Vision Foundation or some other computer vision library. FIG. 6B depicts one embodiment of an annotated image 650. Annotating the image 600 may include selecting a closed contour 652. The selection may be based on selecting the closed contour 652 with the greatest area. The closed contour 652 may correspond to an anatomical feature 604 present in the image 600. In one embodiment, the annotation of an image 600 may include polyline annotation in a JSON file or JSON data. The identification module 160 may then parse the JSON file or data into a polygon and convert the polygon into a binary image. The one or more images 600 or their respective annotated images 650 may form the dataset.

In some embodiments, generating the training, validation, and testing datasets (step 502) may include the identification module 160 splitting the dataset into training, validation, and testing datasets. Splitting the dataset may include randomly sorting an image 600 and its annotated image 650 into one of the three subsets (i.e., training, validation, testing). The split may include a ratio of 0.8 training/0.1 validation/0.1 testing. The split may include 0.5 training/0.25 validation/0.25 testing. The split may include 0.7 training/0.1 validation/0.2 testing. The split may include 0.9 training/0.05 validation/0.05 testing. The split may include some other suitable ratio.

In one embodiment, training the computational models 162 (step 504) may include the identification module 160 training one or more of the computational models 162. A computational model 162 may include a machine learning model, an artificial intelligence (AI) model, a statistical model, or some other type of computational model. A machine learning model may include an artificial neural network, a deep learning architecture (e.g., a deep neural network, a deep belief network, deep reinforcement learning, a recurrent neural network, a convolutional neural network, a long-short term memory model, or a transformer), a decision tree, a support-vector machine (SVM), a Bayesian network, a genetic algorithm, or some other machine learning model. A computational model 162 may include an ensemble of computational models. A computational model may include a supervised model or a model that uses supervised learning techniques. In some embodiments, a computational model 162 may include U-Net or U-Net++ provided by the University of Freiburg, or DenseNet.

Training a computational model 162 (step 504) may include pre-training the computational model 162. Pre-training the computational model 162 may include training the computational model 162 on an image database. The image database may include ImageNet provided by Stanford Vision Lab. Pre-training a computational model 162 may help reduce training times. A pre-trained model 162 may yield higher accuracies early in training and may converge to its maximum accuracy shortly thereafter.

Training a computational model 162 (step 504) may include using an iterative pipeline with forward propagation and loss computation followed by backpropagation and validation checks using the training dataset and the validation dataset. Training a computational model 162 may include selecting a batch size, learning rate, or other training configuration. For example, the batch size may include 8, 4, 1, or some other suitable number. The learning rate may include 5e-5, 1e-4, or some other learning rate. In some embodiments, training may include a low learning rate and no normalization. In one embodiment, training a computational model 162 may include performing a hyperparameter search on the model or otherwise tuning the hyperparameters of the computational model 162.

Training the one or more computational models 162 (step 504) may include training multiple computational models 162, evaluating the computational models 162, and selecting the best-performing model 162. Evaluating the computational models 162 may include conducting a grid search for each model 162 and determining their respective training and loss curves. Evaluating the computational models 162 may include computing one or more Sorensen-Dice coefficients. In one embodiment, the Dice score may include twice the number of pixels overlapping between the prediction generating by a computational model 162 and the ground truth (as indicated by the annotated image 650 of the relevant image 600) divided by the total number of segmented pixels across both the prediction and the annotated image 650. Evaluating the computational models 162 may include calculating a pixelwise accuracy, an intersection over the union (IoU), a peak signal-to-noise ratio (PSNR), a receiver operating characteristic (ROC), or an area under a curve (AUC). Evaluating the models may include using a loss function, which may include binary cross-entropy (BCE) or some other loss function.

In one embodiment, the identification module 160 may use one or more trained computational models 162 to generate a prediction based on an input image (step 508). The input image may include a frame from the video feed of the endoscope 110. The video feed may include a live video feed from the endoscopic camera 112 during a procedure. The identification module 160 may receive the video feed (step 506) from the I/O interface 152.

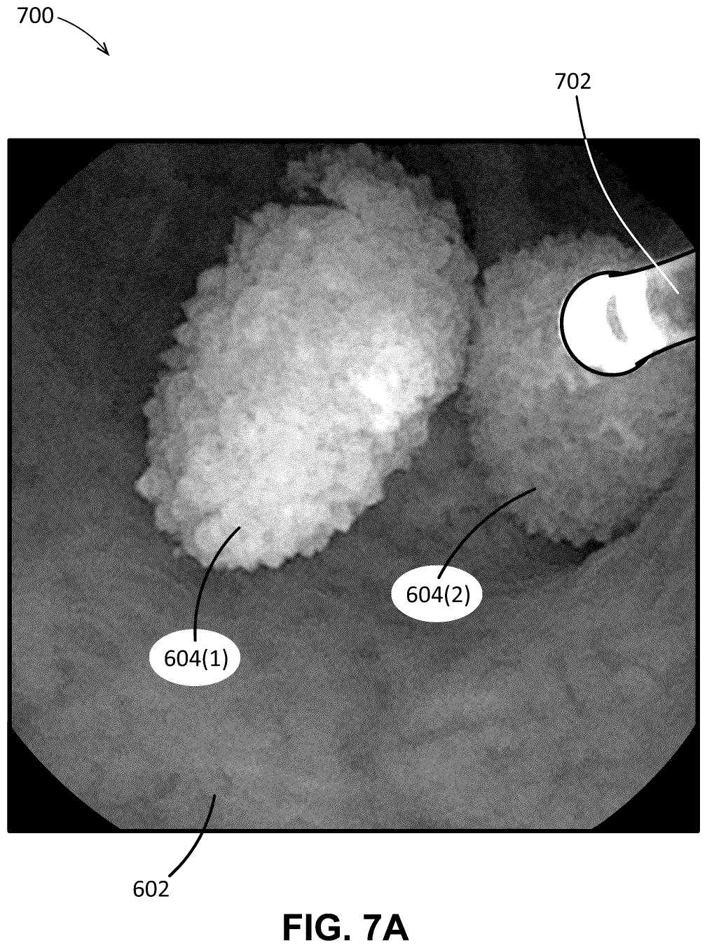

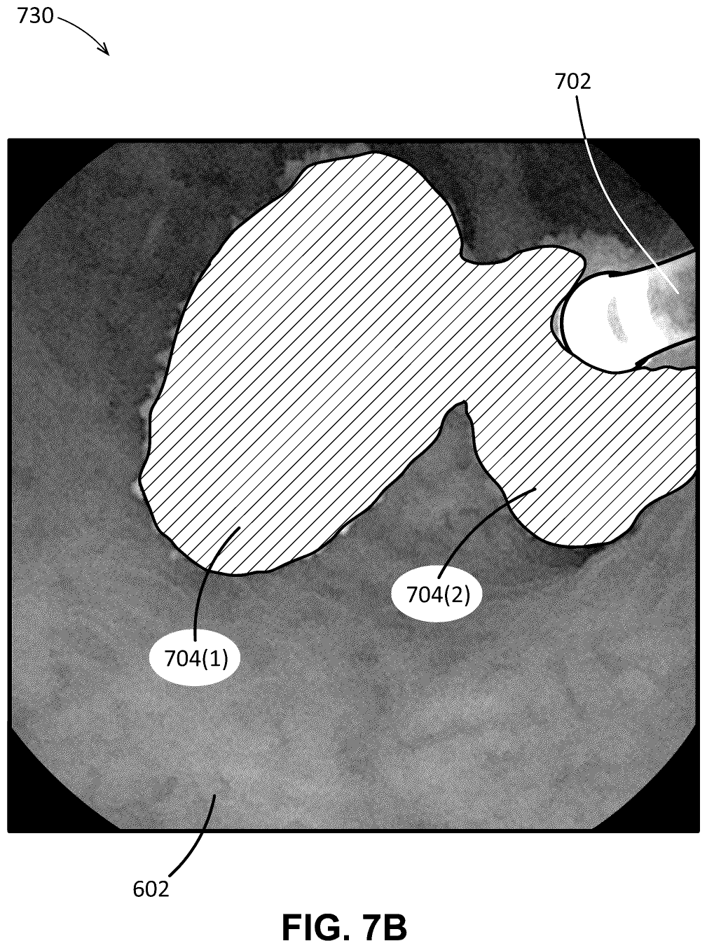

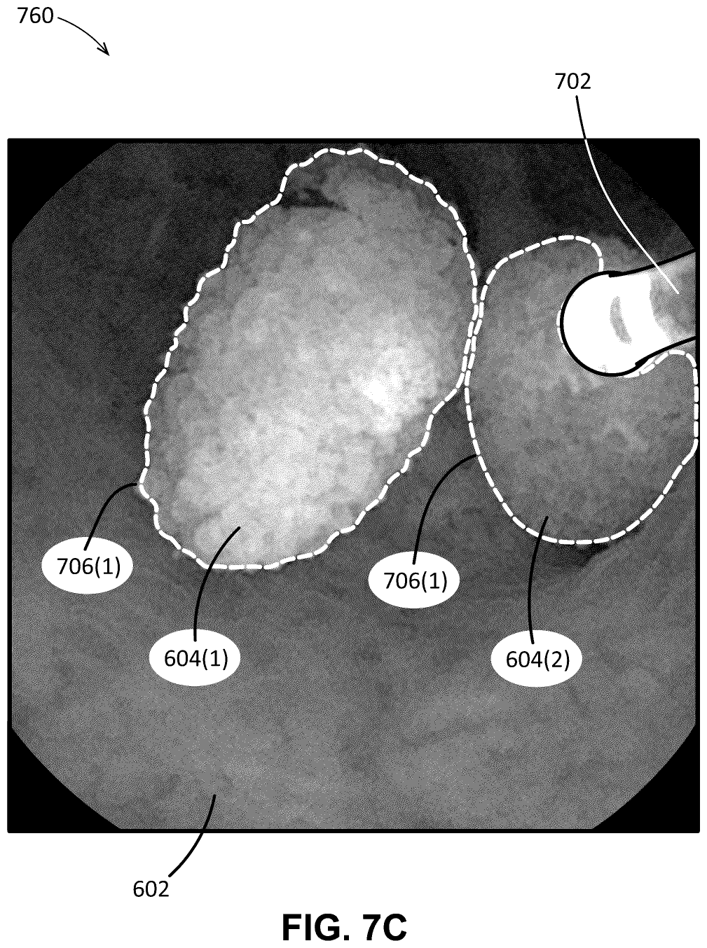

FIG. 7A depicts one embodiment of an input image 700. As can be seen in FIG. 7A, the input image 700 may include a frame of an endoscopic video feed. The input image 700 may include a portion of the anatomical region 602. The input image 700 may include a anatomical feature 604, such as a kidney stone or kidney stone fragment. For example, in FIG. 7A, the input image 700 includes two anatomical features 604(1)-(2), shown here as kidney stones. The input image 700 may include a portion of the endoscope 110, such as a light 702 (which may be one of the instruments 114). The system may accept the input image 700 as input to its one or more trained computational models 162.

Using the one or more trained computational models 162 to generate the prediction (step 508) may include inputting the input image 700 into the computational model 162. The computational model 162 may process the input image 700 and may generate the prediction. The prediction may include data indicating a location in the input image 700 that contains an anatomical feature 604. FIG. 7B depicts one embodiment of a visual representation 730 of a prediction generated by a trained computational model 162. As can be seen in FIG. 7B, the visual representation of the prediction 730 may include an annotation 704 (e.g., image annotation data, an annotated image, etc.) that indicates where in the input image 700 the anatomical feature 604 is located. For example, as can be seen in FIG. 7B, the computational model 162 has identified the two anatomical features 604(1)-(2) and has marked them using annotations 704(1)-(2). The prediction generated by the computational model 162 may include data indicating a contour of the annotation 704, data indicating which pixels are located within the contour of the annotation 704, or other data indicating where, in the input image 700, the computational model 162 has predicted the presence of an anatomical feature 604.

In some embodiments, the prediction 730 of the computational model 162 may be used to adjust a visual display 134 that displays the video feed of the endoscope 110 during surgery (step 510). FIG. 7C depicts one embodiment of a frame 760 from a video feed of an endoscope 110 that has been adjusted based on the prediction 730 generated by the computational model 162. As can be seen in FIG. 7C, the frame 760 may display the anatomical region 602, the anatomical features 604(1)-(2), and the light 702 of the endoscope 110. The visual display 134 may be adjusted to include one or more overlays 706(1)-(2) indicating the location of the one or more anatomical features 604(1)-(2). An overlay 706 may be based on an annotation 704 of a prediction 730. In this example, the overlays 706 each include an outline of the respective anatomical feature 604.

In some embodiments, the overlay 706 may take on a variety of shapes, colors, or other visual characteristics. For example, as depicted in FIG. 7C, the overlay 706 may include an outline of the anatomical feature 604. The outline may include one or more colors that may make the outline easy for the operator to see on the visual display 134. The color may include a color selected by the operator or other user of the computing system 130. The color may include a color that usually contrasts well with the anatomical region 602 or a anatomical feature 604. The color may automatically adjust based on surrounding colors in the visual display 134. In one embodiment, the overlay 706 may include an outline of the anatomical feature 604 that is filled in. The filled in portion may include a solid color, a pattern, or some other visual characteristic. The filled in portion may be at least semi-transparent to allow the operator to still view the anatomical feature 604. In one embodiment, the overlay 706 may include a predetermined icon that indicates the presence of the anatomical feature 604.

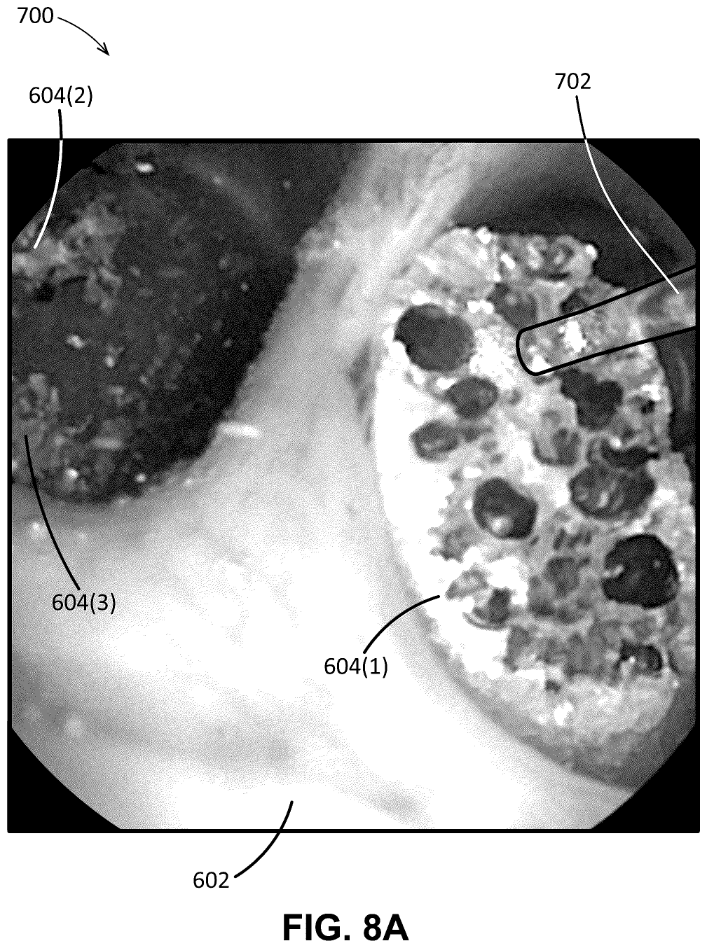

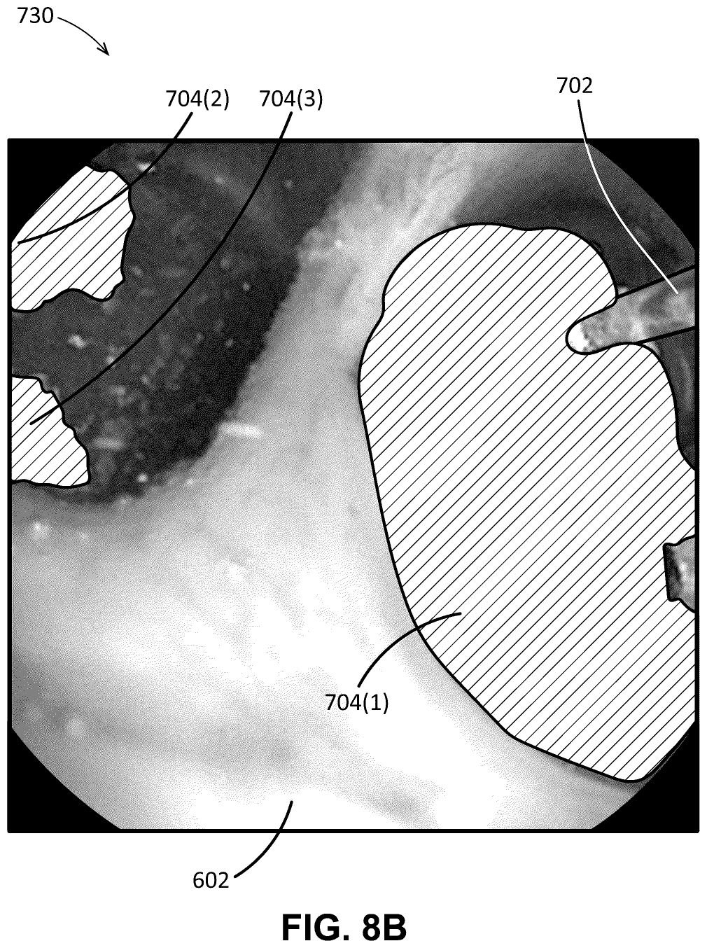

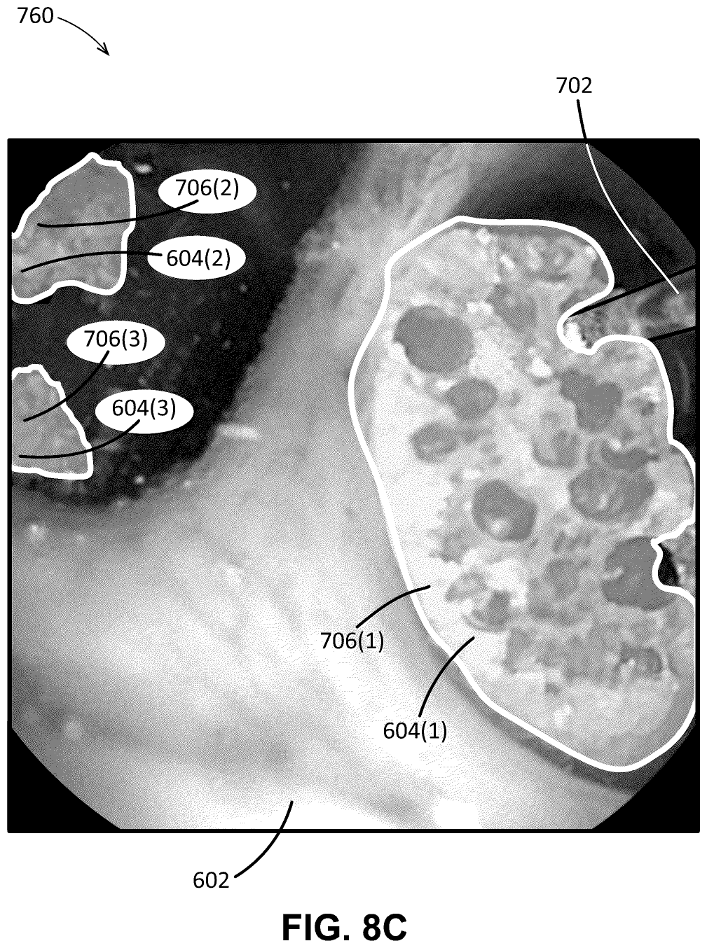

FIGS. 8A-8C depict another example of an input image 700, visual representation of a prediction 730, and a frame 760. As can be seen from FIG. 8A, the input image 700 may include a anatomical feature 604(1) that is in the foreground and, thus, appears to be large relative to one or more anatomical features 604(2)-(3) in the background, which may be smaller than the anatomical feature 604(1) due to being fragments or due to being farther away from the endoscope's 110 camera 112. Nevertheless, as shown in FIG. 8B, the one or more computational models 162 may still identify the anatomical features 604(1)-(3) as indicated by the annotations 704(1)-(3). The frame 760 of FIG. 8C may display overlays 706(1)-(3) for each anatomical feature 604(1)-(3). As can be seen in FIG. 8C, an overlay 706 may include a colored area in the shape of the annotation 704 for the corresponding anatomical feature 604. Furthermore, the overlay 706 may be semitransparent, which may both (1) indicate to the operator the presence and shape of the anatomical feature 604 and (2) allow the operator to still view the underlying anatomical feature 604.

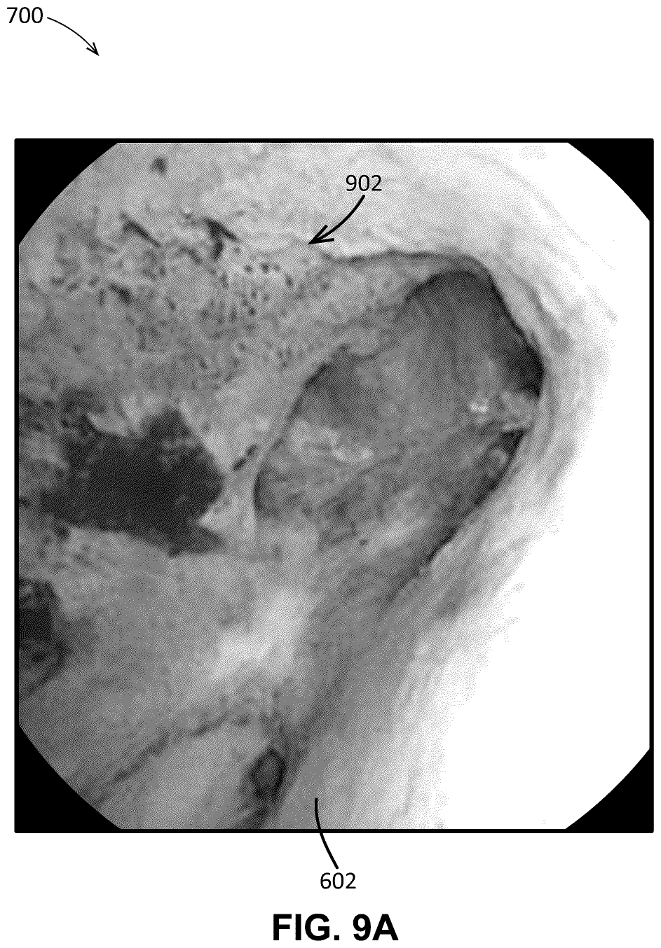

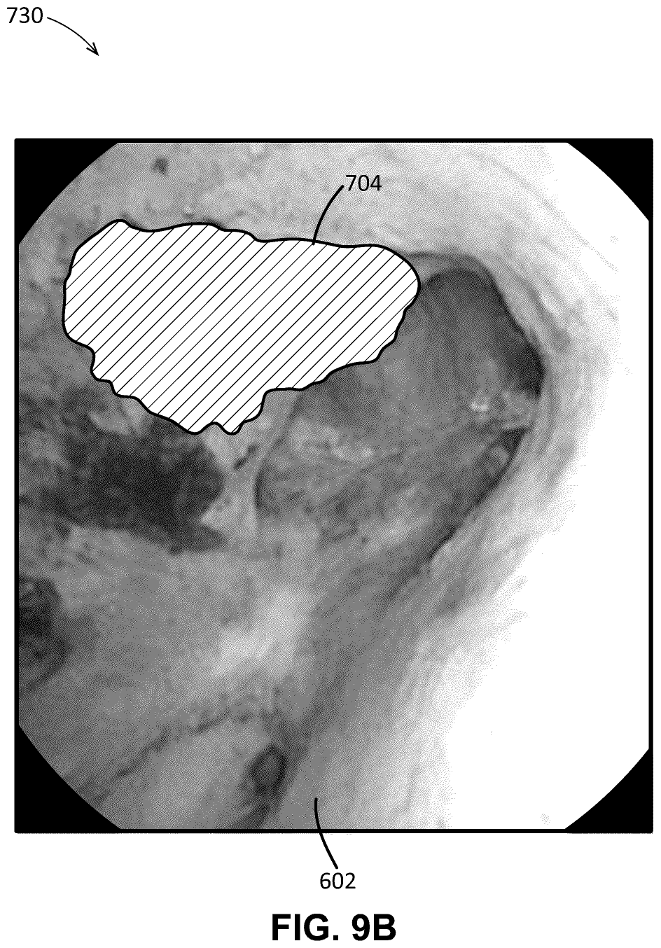

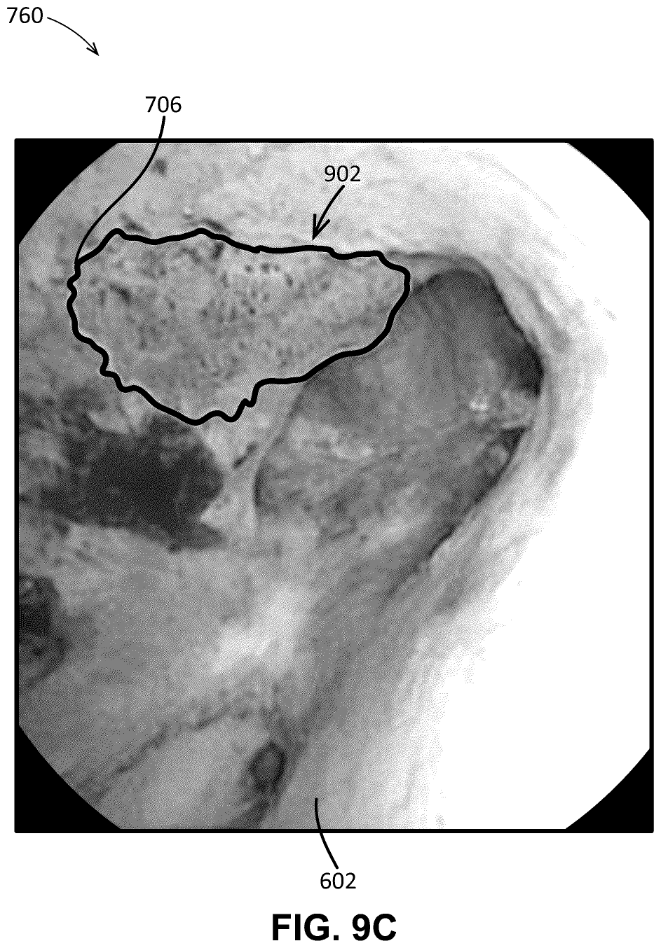

FIGS. 9A-9C depict another example of an input image 700, visual representation of a prediction 730, and a frame 760. In the embodiments shown in FIGS. 9A-9C, instead of the identification module 160 identifying anatomical features 604 in the form of kidney stones or kidney stone fragments, the identification module 160 may identify anatomical features in the form of tumors. One type of tumor the identification module 160 may identify include UTUC tumors. As can be seen from FIG. 9A, in one embodiment, an input image 700 may include a Anatomical feature 902 in the form of a UTUC tumor among the anatomical region 602 (in the form of a patient's collecting system). The one or more computational models 162 may have been trained on a training dataset including such anatomical features 902 (tumors) such that the one or more models 162 can predict the presence of such anatomical features 902 in an input image 700. FIG. 9B depicts a visual representation 730 of a prediction, including an annotation 704 indicating the presence of an anatomical feature 902. FIG. 9C depicts one example frame 760 that may display an overlay 706 over the identified anatomical feature 902. As can be seen in FIG. 9C, the overlay 706 may include a colored outline in the shape of the annotation 704 for the corresponding kidney stone or fragment 604. Furthermore, the overlay 706 may be transparent except for the perimeter of the overlay 706, which may both (1) indicate to the operator the presence and shape of the anatomical feature 902 and (2) allow the operator to still view the underlying anatomical feature 902.

Because the video feed of the endoscope 110 constantly provides sequential frames to the one or more computational models 162 as input images 700 (including, in some embodiments, in real time or near real time), and because the one or more models 162 constantly generate predictions based on the input images 700, the video displayed to the operator with the overlays 706 based on the predictions of the computational models 162, the video with the overlays 706 may also constantly update (including, in some embodiments, in real time or near real time) such that, as the operator moves the endoscope 110 to get a different view, the overlays 706 may follow the anatomical features 604 in the visual display 134. Similarly, in response to the operator performing the surgery and the anatomical feature 604 either moving or fragmenting (as may be the case with kidney stones), the overlays 706 of the video may constantly update and follow the moved or fragmented anatomical feature 604 since the computational models 162 continue to identify the moved or fragmented anatomical feature 604 based on the input images 700 of the endoscope's 110 video feed.

In some embodiments, the computing system 130 may display both the video feed with the overlay 706 and the video feed without the overlay 706 on one or more visual displays 134. The two video feeds may display side by side and may be synchronized such that they show the video, one with the overlay 706 and one without. This may allow the operator to have both an unobstructed view of the video feed of the endoscope 110 and a view that indicates the locations and presence of anatomical features 604.

In one or more embodiments, the one or more computational models 162 may accept, as input, multiple input images 700. The multiple input images 700 may include one or more frames of the endoscope 110 video feed. The one or more frames may include sequential frames from the video feed. This may allow for more consistent predictions between subsequent frames. This may allow the one or more models 162 to account for memory of data from previous input images 700 without the overhead of additional input dimensions. In some embodiments, the one or more models 162 may incorporate self-attenuation modules, which may increase performance in video segmentation.

In some embodiments, the dataset used to create the training, validation, and testing datasets may include images of other anatomical phenomena such that the one or more computational models 162 may be capable of multi-class segmentation. Thus, the one or more models 162 may be able to identify healthy tissues over unhealthy tissue, clots, tumors (e.g., Anatomical features 902, as described above regarding FIGS. 9A-9C) or other objects or phenomena encountered during surgery.

Accordingly, the present disclosure provides for a method of navigating an anatomical region, which may include identifying and tracking an anatomical feature of an anatomical region. As discussed above, identifying and tracking the anatomical feature of the anatomical region may include generating training, validation, and testing datasets, wherein the training, validation, and testing datasets each include a plurality of images, where one or more images of the plurality of images are annotated with a location of the anatomical feature of the anatomical region in the image. Such identifying an tracking may further include training a computational model, which may include steps such as training the computational model on the training dataset using forward propagation and loss computation to adjust one or more parameters of the computational model, validating the computational model on the validation dataset, receiving a frame of a video feed from an endoscope, and inputting the frame into the computational model. Further, such identifying and tracking may include generating, via the computational model, an output, where the output includes data indicating one or more locations of the frame containing the anatomical feature, and adjusting a visual display that includes the frame with an overlay based on the data indicating one or more locations of the frame containing the anatomical feature.

### Further Applications

While the systems and methods disclosed above have been largely described in relation to surgeries on a patient's collecting system, renal system, and other similar anatomy, the systems and methods described herein can be applied to other types of surgeries. For example, the systems and methods could be applied to surgeries involving a patient's bladder, gastrointestinal system, or trachea. The systems and methods could be applied to other types of surgeries that involve the use of an endoscope 110.

While many of the systems and methods disclosed above have been largely described in relation to three-dimensional mapping, the systems and methods may be applicable to generating and navigating from a two-dimensional mapping of an anatomical region. For example, a two-dimensional mapping of an anatomical region may include a top-down view of a plane intersecting the anatomical region. The two-dimensional map may include the icon indicating the position and orientation of the tip of the endoscope 110. The two-dimensional map may include one or more icons indicating locations of anatomical features within the anatomical region, such as kidney stones and fragments, tumors, or other phenomena within a patient's collecting system. The two-dimensional map may include one or more arrows that provide navigational directions to one or more of the anatomical features. In some embodiments, the two-dimensional map may adjust to show different portions of the anatomical region. For example, the two-dimensional map may translate in one or more of the six degrees of freedom (forward, back, left, right, up, down) or rotate along one or more of the three axes (roll, pitch, yaw). Such adjusting may be relative to the endoscope tip 110 or relative to some other location.

### Hardware and Software Systems and Methods

While the making and using of various embodiments of the present disclosure are discussed in detail herein, it should be appreciated that the present disclosure provides many applicable inventive concepts that are embodied in a wide variety of specific contexts. The specific embodiments discussed herein are merely illustrative of specific ways to make and use the disclosure and do not delimit the scope of the disclosure. Those of ordinary skill in the art will recognize numerous equivalents to the specific apparatuses, systems, and methods described herein. Such equivalents are considered to be within the scope of this disclosure and may be covered by the claims.

Furthermore, the described features, structures, or characteristics of the disclosure may be combined in any suitable manner in one or more embodiments. In the description contained herein, numerous specific details are provided, such as examples of programming, software, user selections, hardware, hardware circuits, hardware chips, or the like, to provide understanding of embodiments of the disclosure. One skilled in the relevant art will recognize, however, that the disclosure may be practiced without one or more of the specific details, or with other methods, components, materials, apparatuses, devices, systems, and so forth. In other instances, well-known structures, materials, or operations may not be shown or described in detail to avoid obscuring aspects of the disclosure.

These features and advantages of the embodiments will become more fully apparent from the description and appended claims, or may be learned by the practice of embodiments as set forth herein. As will be appreciated by one skilled in the art, aspects of the present disclosure may be embodied as an apparatus, system, method, computer program product, or the like. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present disclosure may take the form of a computer program product embodied in one or more computer-readable media having program code embodied thereon.

In some embodiments, a module may be implemented as a hardware circuit comprising custom (very large-scale integration) VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the program code may be stored and/or propagated on in one or more computer-readable media.

In some embodiments, a module may include a smart contract hosted on a blockchain. The functionality of the smart contract may be executed by a node (or peer) of the blockchain network. One or more inputs to the smart contract may be read or detected from one or more transactions stored on or referenced by the blockchain. The smart contract may output data based on the execution of the smart contract as one or more transactions to the blockchain. A smart contract may implement one or more methods or algorithms described herein.

The computer program product may include a computer-readable storage medium (or media) having computer-readable program instructions thereon for causing a processor to carry out aspects of the present disclosure. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer-readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present disclosure.

Aspects of the present disclosure are described herein with reference to flowchart illustrations or block diagrams of methods, apparatuses, systems, algorithms, or computer program products according to embodiments of the disclosure. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The schematic flow chart diagrams included herein are generally set forth as logical flow chart diagrams. As such, the depicted order and labeled steps are indicative of one embodiment of the presented method. Other steps and methods may be conceived that may be equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The schematic flowchart diagrams and/or schematic block diagrams in the Figures illustrate the architecture, functionality, and operation of possible implementations of apparatuses, systems, methods and computer program products according to various embodiments of the present disclosure. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

Although the devices, systems and methods set forth in this disclosure and accompanying figures are described for use in kidney surgery in some embodiments, the devices, systems and methods may be employed for use in other types of surgical or diagnostic procedures involving other parts of human or animal anatomy in various embodiments.

Thus, although there have been described particular embodiments of the present disclosure of new and useful systems and methods for navigation and identification during endoscopic kidney surgery, it is not intended that such references be construed as limitations upon the scope of this disclosure.

What is claimed is:

1. A method of navigating an anatomical region, comprising:
generating a map of an internal space of an anatomical region, including:
segmenting the anatomical region from a preoperative computerized tomography (CT) scan,
generating a three-dimensional reconstruction of the anatomical region from video received from an endoscope, and
registering the three-dimensional reconstruction to the segmented CT scan to generate a three-dimensional map of the anatomical region;
tracking a tip of the endoscope within the anatomical region, including:
initializing a location and orientation of the tip of the endoscope on the three-dimensional map of the anatomical region,
receiving endoscopic video from the endoscope, and
updating the location and orientation of the tip of the endoscope based on applying one or more localization and three-dimensional reconstruction techniques to the received endoscopic video; and
identifying and tracking an anatomical feature of the anatomical region, including:
receiving a frame of the endoscopic video from the endoscope,
inputting the frame into a trained computational model,
generating, via the computational model, an output, wherein the output includes data indicating one or more locations of the frame containing the anatomical feature of the anatomical region, and
adjusting a visual display that includes the frame with an overlay based on the data indicating one or more locations of the frame containing the anatomical feature of the anatomical region.

2. The method of claim 1, wherein the anatomical region is a collecting system of a kidney, and the anatomical feature of the anatomical region is a kidney phenomenon.

3. The method of claim 2, wherein the kidney phenomenon is one of:
a kidney stone;
a kidney stone fragment; or
a tumor.

4. The method of claim 2, wherein generating the three-dimensional reconstruction of the anatomical region includes using one or more localization and three-dimensional reconstruction techniques.

5. The method of claim 4, wherein the one or more localization and three-dimensional reconstruction techniques includes at least one of:
structure from motion; or
simultaneous localization and mapping (SLAM).

6. The method of claim 5, wherein the computational model is trained by:
generating training, validation, and testing datasets, wherein the training, validation, and testing datasets each include a plurality of images, wherein one or more images of the plurality of images are annotated with a location of one or more anatomical features in the image;
training the computational model on the training dataset using forward propagation and loss computation to adjust one or more parameters of the computational model; and
validating the computational model on the validation dataset.

7. The method of claim 6, wherein generating the training, validation, and testing datasets includes:
receiving the plurality of images; and
for each image in the plurality of images,
using a computer vision library to determine one or more contours,
selecting a closed contour of the one or more contours, and
annotating the image based on a bounding box associated with the selected closed contour.

8. The method of claim 6, wherein validating the computational model on the validation dataset includes computing one or more Sorenson-Dice coefficients.

9. The method of claim 6, wherein the computational model includes at least one of:
U-Net;
U-Net++; or
DenseNet.

10. A method of navigating an anatomical region, comprising:
generating a map of an internal space of an anatomical region, including:
segmenting the anatomical region from a preoperative computerized tomography (CT) scan,
generating a three-dimensional reconstruction of the anatomical region from video received from an endoscope, and
registering the three-dimensional reconstruction to the segmented CT scan to generate a three-dimensional map of the anatomical region; and
tracking a tip of the endoscope within the anatomical region, including:
initializing a location and orientation of the tip of the endoscope on the three-dimensional map of the anatomical region,
receiving endoscopic video from the endoscope, and
updating the location and orientation of the tip of the endoscope based on applying one or more localization and three-dimensional reconstruction techniques to the received endoscopic video.

11. The method of claim 10, wherein the anatomical region is a collecting system of a kidney.

12. The method of claim 11, wherein generating the three-dimensional reconstruction of the anatomical region includes using one or more localization and three-dimensional reconstruction techniques.

13. The method of claim 12, wherein the one or more localization and three-dimensional reconstruction techniques includes structure from motion.

14. The method of claim 12, wherein the one or more localization and three-dimensional reconstruction techniques includes simultaneous localization and mapping (SLAM).

15. A method of identifying and tracking an anatomical feature of an anatomical region, comprising:
generating training, validation, and testing datasets, wherein the training, validation, and testing datasets each include a plurality of images, wherein one or more images of the plurality of images are annotated with a location of one or more anatomical features of an anatomical region in the image;
training a computational model, including:
training the computational model on the training dataset using forward propagation and loss computation to adjust one or more parameters of the computational model, and
validating the computational model on the validation dataset;
receiving a frame of a video feed from an endoscope;
inputting the frame into the computational model;
generating, via the computational model, an output, wherein the output includes data indicating one or more locations of the frame containing an anatomical feature; and
adjusting a visual display that includes the frame with an overlay based on the data indicating one or more locations of the frame containing the anatomical feature.

16. The method of claim 15, wherein the anatomical region is a collecting system of a kidney, and the feature of the anatomical region is a kidney phenomenon.

17. The method of claim 16, wherein the kidney phenomenon is one of:
a kidney stone;
a kidney stone fragment; or
a tumor.

18. The method of claim 16, wherein generating the training, validation, and testing datasets includes:
receiving the plurality of images; and
for each image in the plurality of images,
using a computer vision library to determine one or more contours,
selecting a closed contour of the one or more contours, and
annotating the image based on a bounding box associated with the selected closed contour.

19. The method of claim 16, wherein validating the computational model on the validation dataset includes computing one or more Sorenson-Dice coefficients.

20. The method of claim 16, wherein the computational model includes at least one of:
U-Net;
U-Net++; or
DenseNet.
